(19) United States
(12) Patent Application Publication
(10) Pub. No.: US 2026/0001854 A1
(43) Pub. Date: Jan. 1, 2026

(54) CONJUGATE COMPOUND COMPRISING 13-CIS-RETINOIC ACID AND ASCORBIC ACID

(71) Applicant: Mastery Biotech CO., LTD., Taipei City (TW)

(72) Inventor: Bo-Lin Lin, Taipei City (TW)

(21) Appl. No.: 19/321,865

(22) Filed: Sep. 8, 2025

Related U.S. Application Data
(63) Continuation of application No. PCT/US2024/019035, filed on Mar. 8, 2024.
(60) Provisional application No. 63/489,286, filed on Mar. 9, 2023.

(51) Int. Cl. C07D 307/62; A61K 31/375; A61P 35/00

(52) U.S. Cl. CPC C07D 307/62; A61K 31/375; A61P 35/00

(57) ABSTRACT

The present invention relates to a conjugate compound comprising 13-cis-retinoic acid and vitamin C, and its pharmaceutically acceptable salts thereof. The present invention also relates to a pharmaceutical composition comprising the compound and a pharmaceutically acceptable carrier. The present compound has a much better water solubility than that of retinoic acid, and thus reduces an effective dosage of retinoic acid and minimizes side effects. The present compound increases the efficiency and concentration of 13-cis-retinoic acid to cross the cell membrane into the cell. The present compound is useful in treating cancer such as pancreatic cancer.

### CROSS-REFERENCE TO RELATED APPLICATION(S)

This application is a continuation of PCT/US2024/019035, filed Mar. 8, 2024; which claims the benefit of U.S. Provisional Application No. 63/489,286, filed Mar. 9, 2023. The contents of the above-identified applications are incorporated herein by reference in their entireties.

### FIELD OF THE INVENTION

The present invention relates to a conjugate compound comprising 13-cis-retinoic acid and vitamin C, and its pharmaceutically acceptable salts thereof. The present invention also relates to a pharmaceutical composition comprising the compound and a pharmaceutically acceptable carrier.

### BACKGROUND OF THE INVENTION

All-trans retinoic acid (ATRA), also called tretinoin, and isotretinoin (13-cis-Retinoic acid) are orally active vitamin A derivatives. 13-Cis-retinoic acid and ATRA are promising compounds for treatment of a variety of cancers because of its specific effects on cell proliferation, differentiation, and apoptosis, as well as its low toxicity. Retinoic acid receptors in human cell nuclei have been discovered by biochemists and found not mutated in cancer cells, and thus, retinoic acids could potentially exert its anticancer effects in many malignancies. For example, it was found that in children with high-risk neuroblastoma, treatment with 13-cis-retinoic acid can reduce the risk of the cancer coming back after high-dose chemotherapy and stem cell transplant. ATRA has been studied in combination with other drugs in various cancers and precancerous lesions. A number of clinical trials using ATRA as a part of combination therapy are currently underway [Kocher, H. M., Basu, B., Froeling, F. E. M. et al. Phase I clinical trial repurposing all-trans retinoic acid as a stromal targeting agent for pancreatic cancer. Nat Commun 11, 4841 (2020)]. For instance, ATRA with different interferons (IFN) have been shown to enhance the effects of both drugs and lead to growth inhibition and cell death in tumor cell lines. Nevertheless, to unleash the therapeutic potentials of retinoic acids, many studies emphasize the need for better understanding of the mechanisms that block retinoic acid signaling and retinoic acid regulated gene expression in cancer, such as acute myeloid leukemia (AML). Combinatorial therapies targeting multiple gene silencing mechanisms may be the most effective strategy in reactivating ATRA-sensitive gene expression and differentiation of AML cells, as well as mediating anticancer activities of ATRA in general. Currently, the identification of classes of proteins that control gene expression via histone and DNA modifications is driving the development of new therapeutic agents, so-called epigenetic drugs that alter chromatin structure. However, these epigenetic modifying drugs have been shown to be only partly effective against different cancers when used alone.

Vitamin A is a fat-soluble vitamin and an essential nutrient for humans. It is a group of organic compounds that includes retinol, retinal (also known as retinaldehyde), retinoic acid, and several provitamin A carotenoids (most notably beta-carotene [β-carotene]). Retinoic acid is a metabolite of vitamin A1 (all-trans-retinol). Isotretinoin (13-cis-retinoic acid) is an orally active vitamin A derivative.

Vitamin C (also known as ascorbic acid and ascorbate) is a water-soluble vitamin found in citrus and other fruits and vegetables. Vitamin C is an essential nutrient involved in the repair of tissue, the formation of collagen, and the enzymatic production of certain neurotransmitters. Vitamin C is required for the functioning of several enzymes and is important for immune system function. Vitamin C also functions as an antioxidant.

### BRIEF DESCRIPTION OF THE DRAWINGS

FIG. 1 shows examples of synthesizing the conjugate compound of Formula A1 and A2 without a protecting group.

FIG. 2 shows examples of synthesizing the conjugate compound of Formula A2 with a protecting group. Protecting group 2-methoxyethoxymethyl ether (MEM) is used as an illustration. Other protecting groups may also be used.

FIG. 3. shows an example of synthesizing the conjugate compound of Formula A1 used in this study.

### DETAILED DESCRIPTION OF THE INVENTION

### Definitions

“Pharmaceutically acceptable salts,” as used herein, are salts that retain the desired biological activity of the parent compound and do not impart undesired toxicological effects. Pharmaceutically acceptable salt forms include various crystalline polymorphs as well as the amorphous form of the different salts. The pharmaceutically acceptable salts can be formed with metal or organic counterions and include, but are not limited to, alkali metal salts such as sodium or potassium; alkaline earth metal salts such as magnesium or calcium; and ammonium or tetraalkyl ammonium salts, i.e., NX4+ (wherein X is C1-4).

### Abbreviations

HATU, 1-[Bis(dimethylamino)methylene]-1H-1,2,3-triazolo[4,5-b]pyridinium 3-oxide hexafluorophosphate
DIPEA, N,N-Diisopropylethylamine
DMAP, 4-Dimethylaminopyridine
DCC, N,N′-Dicyclohexylcarbodiimide
NHS, N-Hydroxysuccinimide
THF, Tetrahydrofuran
DMF, Dimethylformamide
Ac, Acetate
ACN, Acetonitrile
Bn, Benzyl ether
BOM, Benzyloxymethyl acetal
Bz, Benzoate
MEM, 2-Methoxyethoxymethyl ether
MEMCI, 2-Methoxyethoxymethyl chloride
MOM, Methoxymethyl acetal
MOP, Methoxypropyl acetal
Nap, 2-Naphthylmethyl ether
NMR, Nuclear Magnetic Resonance
PMB, 4-Methoxybenzyl ether
TBDMS, Tert-butyldimethylsilyl ether
TBDPS, Tert-butyldiphenylsilyl ether
TBS, Tert-butyldimethylsilyl ether
TES, Triethylsilyl ether
THP, Tetrahydropyranyl acetal
TIPS, Triisopropylsilyl ether
TMS, Trimethylsilyl ether
Troc, 2,2,2-Trichloroethyl carbonate

### Conjugate Compound Comprising 13-Cis-Retinoic Acid and Ascorbic Acid

The present invention relates to conjugate compounds of Formula A1 and A2 comprising 13-cis-retinoic acid and vitamin C (ascorbic acid). The 13-cis-retinoic acid in the conjugate compound provides therapeutic activity, and vitamin C in the conjugate compound increases the water solubility of the compound, and significantly reduces the effective dosage of 13-cis-retinoic acid in the compound for treating a disease.

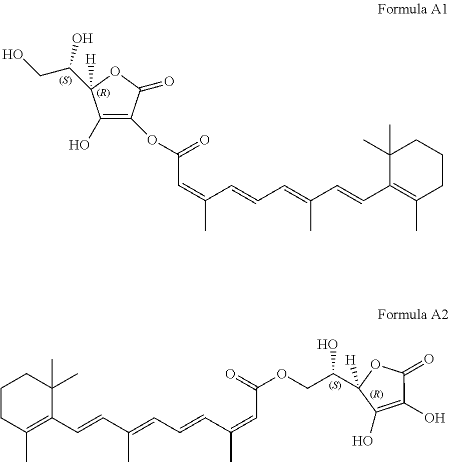

### Preparation of Formula A1 and Formula A2 Compounds

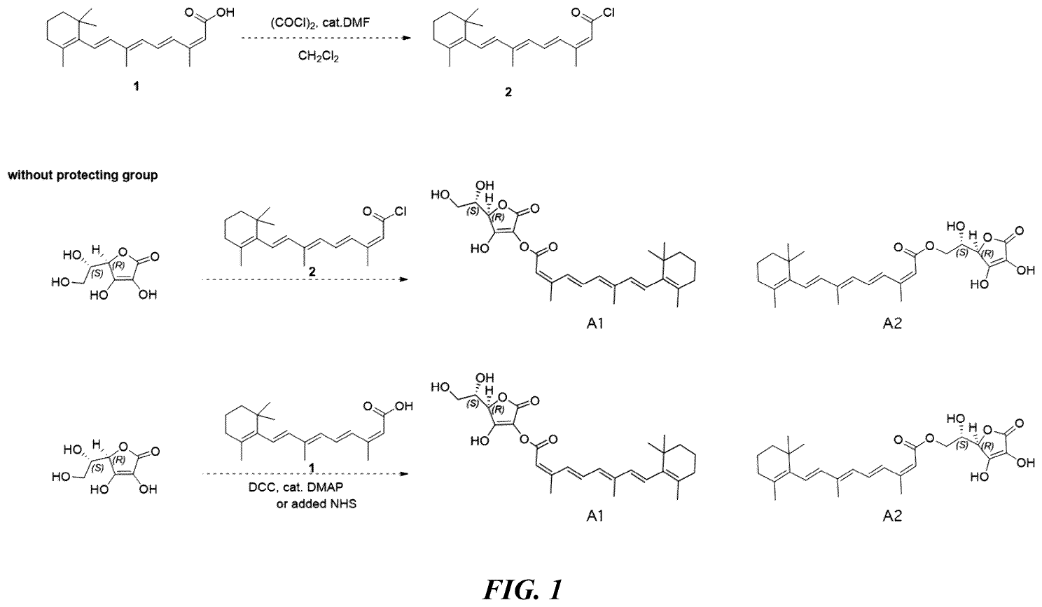

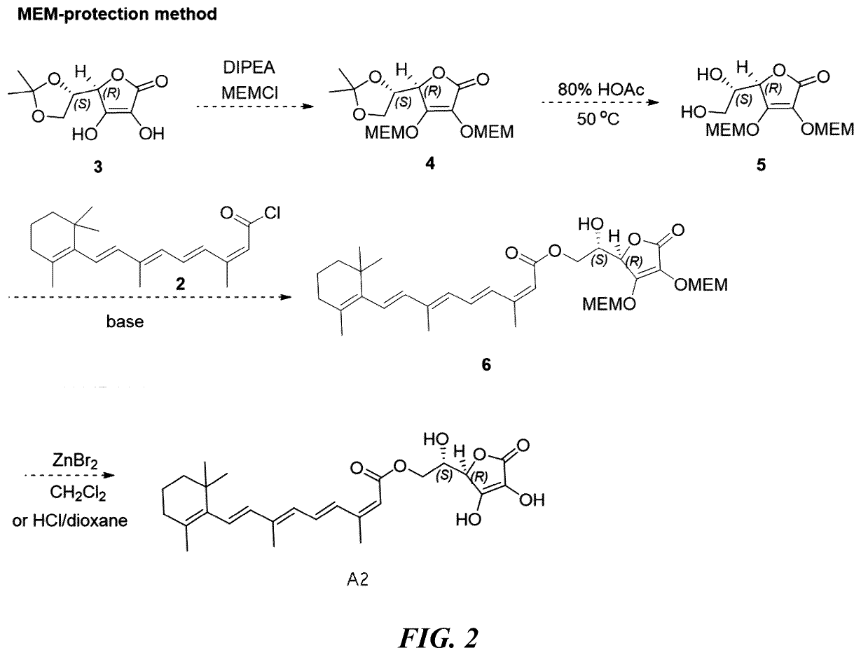

FIGS. 1 and 2 show examples of different schemes (with or without a protecting group) of synthesizing the conjugate compound of Formula A1 and A2.

As shown in FIG. 1, for forming retinoic acid chloride (compound 2), a solution of retinoic acid (1) is added with (COCl)₂ and CH₂Cl₂ in a catalytic amount of dimethylformamide (DMF) under stirring at room temperature. Subsequently, distillation is carried out under reduced pressure to remove unreacted (COCl)₂ and DMF, to obtain retinoic acid chloride (compound 2).

In some embodiments, the compounds represented by Formula A1 and Formula A2 can be synthesized directly from combining retinoic acid or retinoic acid chloride with vitamin C (FIG. 1). For example, a solution of vitamin C is mixed with either retinoic acid (compound 1) or retinoic acid chloride (compound 2) in the presence of a catalyst system of N,N′-dicyclohexylcarbodiimide (DCC) and 4-dimethylaminopyridine (DMAP) or added with N-hydroxysuccinimide (NHS) at room temperature. After completion of the reaction, the solution is concentrated under reduced pressure to obtain a target compound in a solid form.

In some embodiments, Formula A2 compound can be synthesized by using a protecting group (FIG. 2). For example, isopropylidene ascorbic acid (compound 3) is dissolved in a dichloromethane solution and then added with N,N-diisopropylethylamine (DIPEA), followed by addition of 2-methoxyethoxymethyl chloride (MEMCI), so as to form compound 4, in which the hydroxyl groups at C2 and C3 in the ascorbic acid ring are protected by the 2-methoxyethoxymethyl ether (MEM) protecting groups. Subsequently, compound 4 is dissolved in 80% ethanoic acid (HOAc) and reacted at 50° C. for 2 hours. After completion of the reaction, the solution is concentrated under reduced pressure to remove ethanoic acid. The residue (compound 5) and retinoic acid chloride (compound 2) prepared are further dissolved in DMF and added with DCC and DMAP at room temperature. The mixture is then stirred for 2 hours to form compound 6.

Thereafter, the protecting group can be removed by treatment with an appropriate deprotecting agent. For example, removal of the MEM protecting groups in compound 6 was achieved by reacting compound 6 with anhydrous zinc bromide (ZnBr₂) in CH₂Cl₂ or HCl/dioxane at room temperature for 2 hours. After completion of the reaction, the solution was concentrated and dried. The residue is then purified to give Formula A2 compound.

Protecting groups suitable for the present methods include, but are not limited to, 2,2,2-trichloroethyl carbonate (Troc), 2-methoxyethoxymethyl ether (MEM), 2-naphthylmethyl ether (Nap), 4-methoxybenzyl ether (PMB), acetate (Ac), benzoate (Bz), benzyl ether (Bn), benzyloxymethyl acetal (BOM), methoxymethyl acetal (MOM), methoxypropyl acetal (MOP), methyl ether, tetrahydropyranyl acetal (THP), triethylsilyl ether (TES), triisopropylsilyl ether (TIPS), trimethylsilyl ether (TMS), tert-butyldimethylsilyl ether (TBS or TBDMS), and tert-butyldiphenylsilyl ether (TBDPS).

### Pharmaceutical Compositions

The present invention provides pharmaceutical compositions comprising one or more pharmaceutically acceptable carriers and a conjugate compound of Formula A1 or A2 or a pharmaceutically acceptable salt thereof.

In one embodiment, the compound is incorporated into any acceptable carrier, including creams, gels, lotions or other types of suspensions that can stabilize the compound and deliver it to the affected area by topical applications. In another embodiment, the pharmaceutical composition can be in a dosage form such as tablets, capsules, granules, fine granules, powders, syrups, suppositories, injectable solutions, patches, or the like. The above pharmaceutical composition can be prepared by conventional methods.

Pharmaceutically acceptable carriers, which are inactive ingredients, can be selected by those skilled in the art using conventional criteria. Pharmaceutically acceptable carriers include, but are not limited to, non-aqueous based solutions, suspensions, emulsions, microemulsions, micellar solutions, gels, and ointments. The pharmaceutically acceptable carriers may also contain ingredients that include, but are not limited to, saline and aqueous electrolyte solutions; ionic and nonionic osmotic agents such as sodium chloride, potassium chloride, glycerol, and dextrose; pH adjusters and buffers such as salts of hydroxide, phosphate, citrate, acetate, borate; and trolamine; antioxidants such as salts, acids and/or bases of bisulfite, sulfite, metabisulfite, thiosulfite, ascorbic acid, acetyl cysteine, cysteine, glutathione, butylated hydroxyanisole, butylated hydroxytoluene, tocopherols, and ascorbyl palmitate; surfactants such as lecithin, phospholipids, including but not limited to phosphatidylcholine, phosphatidylethanolamine and phosphatidyl inositiol; poloxamers and poloxamines, polysorbates such as polysorbate 80, polysorbate 60, and polysorbate 20, polyethers such as polyethylene glycols and polypropylene glycols; polyvinyls such as polyvinyl alcohol and povidone; cellulose derivatives such as methylcellulose, hydroxypropyl cellulose, hydroxyethyl cellulose, carboxymethyl cellulose and hydroxypropyl methylcellulose and their salts; petroleum derivatives such as mineral oil and white petrolatum; fats such as lanolin, peanut oil, palm oil, soybean oil; mono-, di-, and triglycerides; polymers of acrylic acid such as carboxypolymethylene gel, and hydrophobically modified cross-linked acrylate copolymer; polysaccharides such as dextrans and glycosaminoglycans such as sodium hyaluronate. Other pharmaceutically acceptable carriers include xanthan gum, carrageenan, Avicel RC-591 (a combination of microcrystalline cellulose and), and polyethylene glycol. Alternately, the active compound may be dissolved or suspended in a pharmaceutically acceptable lipid formulation such as those described by Kalepu et al (Acta Pharmaceutica Sinica B, 3:361-372, 2013), for example, vegetable oil, coconut oil, castor oil, etc.

Such pharmaceutically acceptable carriers may be preserved against bacterial contamination using well-known preservatives, these include, but are not limited to, benzalkonium chloride, ethylenediaminetetraacetic acid and its salts, benzethonium chloride, chlorhexidine, chlorobutanol, methylparaben, thimerosal, and phenylethyl alcohol, or may be formulated as a non-preserved formulation for either single or multiple use.

For example, a tablet formulation or a capsule formulation of the compound may contain other excipients that have no bioactivity and no reaction with the compound. Excipients of a tablet or a capsule may include fillers, binders, lubricants and glidants, disintegrators, wetting agents, and release rate modifiers. Binders promote the adhesion of particles of the formulation and are important for a tablet formulation. Examples of excipients of a tablet or a capsule include, but not limited to, carboxymethylcellulose, cellulose, ethylcellulose, hydroxypropylmethylcellulose, methylcellulose, karaya gum, starch, tragacanth gum, gelatin, magnesium stearate, titanium dioxide, poly (acrylic acid), and polyvinylpyrrolidone. For example, a tablet formulation may contain inactive ingredients such as colloidal silicon dioxide, crospovidone, hypromellose, magnesium stearate, microcrystalline cellulose, polyethylene glycol, sodium starch glycolate, and/or titanium dioxide. A capsule formulation may contain inactive ingredients such as gelatin, magnesium stearate, and/or titanium dioxide.

For example, a patch formulation of the compound may comprise some inactive ingredients such as 1,3-butylene glycol, dihydroxyaluminum aminoacetate, disodium edetate, D-sorbitol, gelatin, kaolin, methylparaben, polysorbate 80, povidone, propylene glycol, propylparaben, sodium carboxymethylcellulose, sodium polyacrylate, tartaric acid, titanium dioxide, and purified water. A patch formulation may also contain skin permeability enhancer such as lactate esters or diethylene glycol monoethyl ether.

Topical formulations including the compound can be in a form of gel, cream, lotion, liquid, emulsion, ointment, spray, solution, and suspension. The inactive ingredients in the topical formulations for example include, but not limited to, (emollient/permeation enhancer), diethylene glycol monoethyl ether (emollient/permeation enhancer), DMSO (solubility enhancer), silicone elastomer (rheology/texture modifier), caprylic/capric triglyceride, (emollient), octisalate, (emollient/UV filter), silicone fluid (emollient/diluent), squalene (emollient), sunflower oil (emollient), and silicone dioxide (thickening agent).

In one embodiment, the concentration of the conjugate compound comprising 13-cis-retinoic acid and vitamin C or the pharmaceutically acceptable salts thereof in the pharmaceutical composition can be, but not limited to, from 0.1 μM to 10 mM, from 0.1 μM to 1 mM, from 0.1 μM to 500μ, from 0.1μ M to 250μ, from 0.1μ M to 100μ, from 0.1μ M to 50μ, from 1μ M to 10 mM, from 1 μM to 1 mM, from 1 μM to 500 μM, from 1 μM to 250 μM, from 1 μM to 100 μM, from 1 μM to 50 μM, from 10 μM to 10 mM, from 10 μM to 1 mM, from 10 μM to 500 μM, from 10 μM to 250 μM, from 10 μM to 100 μM, or from 10 μM to 50 μM.

### Method of Use

The present conjugate compounds are useful for treating cancer, such as pancreatic cancer, lung cancer, colorectal cancer, ovarian cancer, adrenal cancer, bone cancer, brain cancer, breast cancer, gallbladder cancer, head and neck cancer, kidney cancer, laryngeal cancer, liver cancer, prostate cancer, parathyroid cancer, skin cancer, stomach cancer, and thyroid cancer. Additional examples of cancers suitable to be treated by the present conjugate compounds include cholangiocarcinoma, acute and chronic lymphocytic and granulocytic tumors, adenocarcinoma, adenoma, basal cell carcinoma, cervical dysplasia and in situ carcinoma, Ewing's sarcoma, epidermoid carcinoma, giant cell tumor, glioblastoma multiforme, hairy-cell tumor, intestinal ganglioneuroma, hyperplastic corneal nerve tumor, islet cell carcinoma, Kaposi's sarcoma, leiomyoma, malignant carcinoid, malignant melanoma, malignant hypercalcemia, marfanoid habitus tumor, medullary carcinoma, metastatic skin carcinoma, mucosal neuroma, myeloma, mycosis fungoides, neuroblastoma, osteosarcoma, pheochromocytoma, polycythemia vera, primary brain tumor, small-cell lung tumor, squamous cell carcinoma of both ulcerating and papillary type, hyperplasia, seminoma, soft tissue sarcoma, retinoblastoma, rhabdomyosarcoma, renal cell tumor, topical skin lesion, reticulum cell sarcoma, and Wilm's tumor.

The present method comprises a step of administering to a cancer patient a compound of Formula A1 or Formula A2, in an amount effective to treat cancer. “An effective amount,” as used herein, is the amount effective to treat cancer by ameliorating the pathological condition or reducing the symptoms of cancer.

The pharmaceutical composition of the present invention can be applied by systemic administration or local administration and. Local administration includes topical administration and inhalation. Systemic administration includes oral, parenteral (such as intravenous, intramuscular, subcutaneous or rectal), and other systemic routes of administration. In systemic administration, the active compound first reaches plasma and then distributes into target tissues. Intravenous administration is a preferred route of administration for the present invention.

Dosing of the composition can vary based on the extent of the cancer and each patient's individual response. For systemic administration, plasma concentrations of the active compound delivered can vary; but are generally 1×10⁻¹⁰-1×10⁻⁴ moles/liter, and preferably 1×10⁻⁸-1×10⁻⁵ moles/liter.

In one embodiment, the pharmaceutical composition is administrated intravenously to the subject. The dosage for intravenous bolus injection or intravenous infusion is generally 0.03 to 20 and preferably 0.03 to 10 mg/kg/day.

In one embodiment, the pharmaceutical composition is administrated orally to the subject. The dosage for oral administration is generally at least 0.1 mg/kg/day and less than 100 mg/kg/day. For example, the dosage for oral administration is 0.1-100 or 0.5-50 mg/kg/day, for a human subject. For example, the dosage for oral administration is 20-1000 mg/day or 100-2000 mg/day, and preferably 20-500, 25-200, 50-500, 50-200, 100-600, 100-400, 100-800, 200-800, 400-800, 400-1200, 500-2000, or 800-2000 mg/day for a human subject.

In one embodiment, the pharmaceutical composition is administrated subcutaneously to the subject. The dosage for subcutaneous administration is generally 0.3-20, and preferably 0.3-3 mg/kg/day.

In one embodiment, the pharmaceutical composition is administered by inhalation. Methods of inhalation include liquid instillation, instillation as a pressurized fluid preparation via metered dose inhaler or equivalent, or inhalation of an aerosolized solution via nebulizer, inhalation of dry powder, and directing soluble or dried material into the air stream during mechanical ventilation. The surface concentrations of the active compound delivered via inhalation can vary, but are generally 1×10⁻¹⁰-1×10⁴ moles/liter, and preferably 1×10⁻⁸-1×10⁻⁵ moles/liter.

Those of skill in the art will recognize that a wide variety of delivery mechanisms are also suitable for the present invention.

The present method is useful in treating a mammalian subject, such as humans, horses, and dogs. The present invention is particularly useful in treating humans.

The following examples further illustrate the present invention. These examples are intended merely to be illustrative of the present invention and are not to be construed as being limiting.

### EXAMPLES

### Example 1. Synthesis of Formula A1 Compound

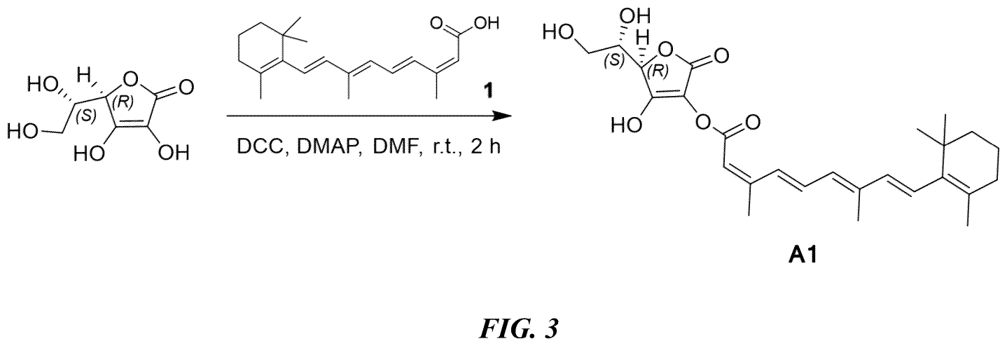

FIG. 3 summarizes one scheme of synthesizing A1 compound.

To a solution of (R)-5-((S)-1,2-dihydroxyethyl)-3,4-dihydroxyfuran-2(5H)-one (1, 200 mg, 1.14 mmol), (2Z,4E,6E,8E)-3,7-dimethyl-9-(2,6,6-trimethylcyclohex-1-en-1-yl)nona-2,4,6,8-tetraenoic acid (2, 409 mg, 1.36 mmol) and DCC (351 mg, 1.70 mmol) in DMF (5 mL) was added DMAP (166 mg, 1.36 mmol). The reaction mixture was stirred at room temperature for 2 hours in a dark environment. After the reaction was completed, the mixture was diluted with water (10 mL) and extracted with EtOAc (10 mL×3). The combined organic layers were concentrated under reduced pressure to obtain a residue. The residue was purified by prep-HPLC (column: Phenomenex luna C18 250 mm×100 mm×10 um; mobile phase: [H₂O (0.1% TFA-H₂O)-ACN]; B%: 20%-50%, 30 min) to give S171-BP (51.65 mg, 9.48% yield) as a yellow solid. All processes were performed in a dark environment with an aluminum foil wrapping. The structure of Formula A1 was confirmed by NMR analysis and summarized below.

MS (ESI): mass calculated for C₂₆H₃₄O₇ 458.55, m/z found 459.45 [M+H]⁺.

¹H NMR (400 MHZ, DMSO-d6): δ 12.52 (br s, 1H), 7.62 (d, J=15.2 Hz, 1H), 7.21-7.14 (m, 1H), 6.35-6.29 (m, 2H), 6.25-6.21 (m, 1H), 5.86 (s, 1H), 5.01-4.78 (m, 3H), 3.80 (t, J=7.0 Hz, 2H), 3.47-3.45 (m, 2H), 1.69 (s, 3H), 2.017 (s, 5H), 1.693 (s, 3H), 1.58-1.56 (m, 1H), 1.45-1.44 (m, 1H), 1.02 (s, 6H).

### Example 2. Cytotoxicity of Formula A1 Compound on Cancer Cell Lines

Objectives: To test the cytotoxicity effect of Formula A1 compounds on three cancer cell lines in vitro.

Cancer Cell Lines

| Cell line | Tumor type^{@} | Culture medium* |
|---|---|---|
| A549 | Non-small Cell Lung Cancer | DMEM |
| PANC-1 | Pancreatic Cancer | DMEM |
| Caco-2 | Colorectal Cancer | DMEM |

^{@}All the cell lines used in this study were derived from human patients.
*All the complete culture media were prepared with fetal bovine serum to a final concentration of 10% and without antibiotics.

Reagents for cell culture and cell viability assay

| Item | Full name |
|---|---|
| DMEM | Dulbecco's Modified Eagle Medium |
| FBS | Fetal bovine serum |
| DPBS | Dulbecco's Phosphate-Buffered Saline |
| Trypsin | 0.25% Trypsin-EDTA |
| DMSO | Dimethyl sulfoxide |
| EtOH | Ethanol |
| MTT | MTT Cell Viability Reagent |

### Procedures

Cells were cultured in indicated growth medium with 10% FBS and were maintained in a humidified incubator at 37° C. containing 5% CO₂. Formula A2 compound was weighted and dissolved in DMSO/EtOH mixture solvent of 1/4 (v/v) ratio to a final concentration of 200 mM at the day of treatment. One day before treatment, cells at log-phase of growth were harvested, counted, and seeded in a 96-well plate at a density of 5×10³ cells/100 mL/well. After cultivation overnight, assumed wells were gently added with 100 mL of fresh media prepared with the serial concentration from 40 to 1000 μM to bring the working concentration from 20 to 500 μM subsequently. The control plate without Formula A2 was carried out as the same procedure as above but 100 mL of fresh media prepared with the equal solvent volume added. All plates were mixed gently and incubated for next 24 hours.

At the day of cell viability detection, tested wells were added 50 mL of MTT working solution and incubated at 37° C. for 3 hours. Spectrophotometric absorbance readings were recorded at wavelength of 540 nm. Readings from blank wells (wells contains only cell culture medium without cells) and negative control wells (wells with cell and culture medium without Formula A2 and solvent treatment) were recorded and used for calibration. After all readings of OD540 subtracted the blank value, the percentage of cell viability were calculated using the following formula:

$$Cell viability ( \% ) = [ OD 540 ( sample ) / OD 540 ( negative control in sample plate ) ] / [ OD 540 ( control ) / OD 540 ( negative control in control plate ) ] \times 100 \%$$

The concentration of Compound A that inhibited cell survival to 50% (IC₅₀) was determined by an online tool “Quest Graph™ IC50 Calculator.” AAT Bioquest, Inc., 26 June. 2024, www.aatbio.com/tools/ic50-calculator.

### Results

The calculated cell viability at different Formula A1 concentrations were listed in Table 1-3. The values were averaged from 6 replicate wells. These cell viability values were further used to estimate the IC50 concentrations for each cell line (Table 4).

TABLE 1
A549 Cell viability vs. Formula A1 concentration

| Formula A1 concentration (μM) | A549 Cell Viability |
|---|---|
| 0 | 100% |
| 20 | 102% |
| 50 | 94% |
| 100 | 100% |
| 150 | 80% |
| 180 | 29% |
| 230 | 63% |
| 253 | 28% |
| 500 | 11% |

TABLE 2
PANC-1 Cell viability vs. Formula A1 concentration

| Formula A2 concentration (μM) | PANC-1 Cell Viability |
|---|---|
| 0 | 100% |
| 20 | 69% |
| 50 | 69% |
| 100 | 54% |
| 150 | 50% |
| 180 | 16% |
| 230 | 17% |
| 253 | 19% |
| 500 | 22% |

TABLE 3
Caco-2 Cell viability vs. Formula A1 concentration

| Formula A2 concentration (μM) | Caco-2 Cell Viability |
|---|---|
| 0 | 100% |
| 200 | 91% |
| 300 | 58% |
| 400 | 33% |
| 500 | 24% |

TABLE 4
IC₅₀ of Formula A1 for A549,
PANC-1 and Caco-2 cell lines

[symbol]

[symbol]

indicates data missing or illegible when filed

TABLE 5
A549 Cell viability vs. 13-cis Retinoic Acid concentration

| 13-cis RA concentration (μM) | A549 Cell Viability |
|---|---|
| 0 | 100% |
| 7.5 | 95% |
| 15 | 78% |
| 30 | 75% |
| 60 | 73% |
| 120 | 57% |
| 240 | 50% |
| 480 | 50% |
| 960 | 51% |

Absolute IC₅₀: 518.23 μM

Using A549 cell line for comparison, the IC₅₀ of 13-cis retinoic acid is 518.23 μM; while the IC₅₀ of Formula A1 is 197.34 μM, indicating that Formula A1 is more effective than 13-cis retinoic acid in inhibiting the growth of A549 cell line.

### Example 3. Synthesis of Formula A2 Compound (Prophetic Example)

Formula A2 is prepared according to FIG. 2.

For forming retinoic acid chloride, a solution of (2Z,4E,6E,8E)-3,7-dimethyl-9-(2,6,6-trimethylcyclohex-1-en-1-yl)nona-2,4,6,8-tetraenoic acid is added with (COCl)₂ and CH₂Cl₂ in a catalytic amount of dimethylformamide (DMF) under stirring at room temperature. Subsequently, distillation is carried out under reduced pressure to remove unreacted (COCl)₂ and DMF, so as to obtain retinoic acid chloride (compound 2).

Formula A2 compound is synthesized by using a protecting group. R)-5-((S)-1,2-dihydroxyethyl)-3,4-dihydroxyfuran-2(5H)-one (1, 500 mg, 1.14 mmol) is dissolved in a dichloromethane solution (5 ml) and then added with N,N-diisopropylethylamine (DIPEA), followed by addition of 2-methoxyethoxymethyl chloride (300 mg, 1.32 mmol) (MEMCI), The reaction mixture is stirred at room temperature for 16 hours in a dark environment. After the reaction is completed, compound 4 is formed and its hydroxyl groups at C2 and C3 in the ascorbic acid ring are protected by the 2-methoxyethoxymethyl ether (MEM) protecting groups.

Subsequently, compound 4 is dissolved in 80% ethanoic acid (HOAc) and reacted at 50° C. for 4 hours. After completion of the reaction, the solution is concentrated under reduced pressure to remove ethanoic acid. Compound 5 and retinoic acid chloride (compound 2) prepared are further dissolved in DMF (5ml) and added with DCC (172 mg, 1.19 mmol) and DMAP (210 mg, 1.36 mmol) at room temperature and stirred for 4 hours to form compound 6. Thereafter, the protecting group is removed by treatment with an appropriate deprotecting agent anhydrous zinc bromide (ZnBr₂) in CH₂Cl₂ or HCl/dioxane at room temperature for 4 hours. After the reaction is completed, the mixture is diluted with water (10 mL) and extracted with EtOAc (10 mL×3). The combined organic layers are concentrated under a reduced pressure to obtain a residue. The residue is purified by prep-HPLC. The residue is then purified to give Formula A2 compound.

It is to be understood that the foregoing describes preferred embodiments of the present invention and that modifications may be made therein without departing from the scope of the present invention as set forth in the claims.

What is claimed is:

1. A compound having the structure of Formula A2 or A1, or a pharmaceutically acceptable salt thereof:

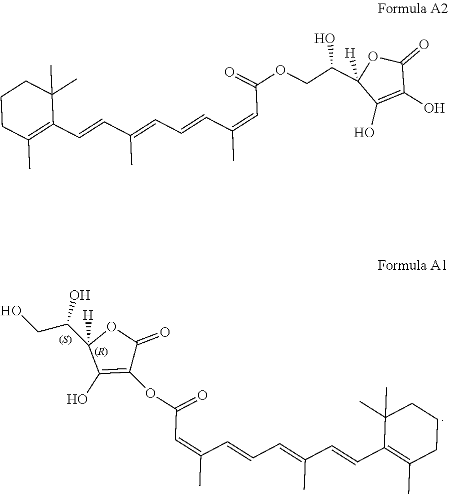

2. The compound according to claim 1, which is Formula A2.

3. The compound according to claim 1, which is Formula A1.

4. A pharmaceutical composition comprising the compound of claim 1, and a pharmaceutically acceptable carrier thereof.

5. A method for treating cancer, comprising administering the compound of claim 1 to a subject in need thereof.

6. The method of claim 3, wherein the compound is administered by intravenous administration.

7. The method of claim 3, wherein the cancer is pancreatic cancer, lung cancer, or colorectal cancer.
